(19) United States
(12) Patent Application Publication
(10) Pub. No.: US 2016/0122194 A1
(43) Pub. Date: May 5, 2016

(54) INTEGRATED PLANT AND METHOD FOR THE FLEXIBLE USE OF ELECTRICITY

(71) Applicant: EVONIK DEGUSSA GMBH, Essen (DE)

(72) Inventors: Georg MARKOWZ, Alzenau (DE); Jürgen Erwin Lang, Karlsruhe (DE); Rüdiger Schütte, Alzenau-Hörstein (DE)

(21) Appl. No.: 14/893,528

(22) PCT Filed: Apr. 30, 2014
(86) PCT No.: PCT/EP2014/058777

§ 371 Date: Nov. 23, 2015

(30) Foreign Application Priority Data
May 28, 2013 (DE) 10 2013 209 882.0

(51) Int. Cl. C01C 3/02; C10L 3/06; C07C 1/12; B01J 19/24; B01J 19/08

(52) U.S. Cl. CPC C01C 3/0208; B01J 19/24; B01J 19/088; C07C 1/12; C10L 3/06; C10L 2290/24; B01J 2219/0805; B01J 2219/24; B01J 2219/00132

(57) ABSTRACT

The present invention relates to an integrated plant which comprises a plant for the electrothermic production of hydrogen cyanide and a separating device for separating hydrogen cyanide from the reaction mixture of the electrothermic production of hydrogen cyanide while obtaining at least one stream of gas containing hydrogen and/or hydrocarbons, the integrated plant having a device for introducing a gas into a natural gas network, to which device a stream of gas containing hydrogen and/or hydrocarbons is fed from the separating device via at least one conduit. This integrated plant affords flexible use of electricity by a method in which a stream of gas, containing hydrogen and/or hydrocarbons, is fed into a natural gas network from the separating device and the amount and/or the composition of the stream of gas fed into the natural gas network is changed in dependence on the electricity supply.

The present invention relates to an integrated plant and a method for the flexible use of electricity.

The use of renewable energy sources, such as wind power, solar energy and hydropower, is gaining ever-increasing significance for the generation of electricity. Electrical energy is typically supplied to a multitude of consumers over long-ranging, supra-regional and transnationally coupled electricity supply networks, referred to as electricity networks for short. Since electrical energy cannot be stored to a significant extent in the electricity network itself or not without further devices, the electrical power fed into the electricity network must be made to match the consumer-side power demand, known as the load. As is known, the load fluctuates time-dependently, in particular according to the time of day, the day of the week and also the time of year. Classically, the load variation is divided into the three ranges, base load, medium load and peak load, and electrical energy generators are, according to type, suitably used in these three load ranges. For a stable and reliable electricity supply, a continuous balance of electricity generation and electricity consumption is necessary. Possibly occurring short-term deviations are balanced out by what is known as positive or negative control energy or control power. In the case of regenerative electricity generating devices, the difficulty arises that, in the case of certain types, such as wind power and solar energy, the energy generating capacity is not available at all times and cannot be controlled in a specific way, but is for example subject to time-of-day and weather-dependent fluctuations, which are only under some circumstances predictable and generally do not coincide with the energy demand at the particular time.

The difference between the generating capacity of fluctuating renewable energy sources and the consumption at a given time usually has to be covered by other power generating plants, such as for example gas, coal and nuclear power plants. With fluctuating renewable energy sources being increasingly extended and covering an increasing share of the electricity supply, ever greater fluctuations between their output and the consumption at the particular time must be balanced out. Thus, even today, not only gas power plants but increasingly also bituminous coal power plants are being operated at part load or shut down in order to balance out the fluctuations. Since this variable operation of the power generating plants involves considerable additional costs, for some time the development of alternative measures has been investigated. As an alternative or in addition to varying the output of a power generating plant, one approach is to adapt the power required by one or more consumers (for example demand-side management, smart grids). Another approach is to store some of the power output when there are high generating outputs from renewable energy sources and retrieve it at times of low generating outputs or high consumption. For this purpose, even today pumped storage power plants are being used for example. Also under development are concepts for storing electricity in the form of hydrogen by electrolytic splitting of water.

The measures described here altogether involve considerable additional costs and efficiency-related energy losses. Against this background, there are increasing attempts to find better possibilities of balancing out the differences between electricity provision and electricity consumption that occur due to the use of renewable energy sources, in particular wind power and solar energy.

An estimated operating time of at most 20%, based on the maximum possible continuous use, results in unacceptably long payback times, so that these plants can only be made profitable by state intervention or applying unusual business models. This estimate is based on the assumption that the plant is only operated at times when there is a surplus from renewable energy sources.

Furthermore, it should be stated that, for the case where there is a low supply of renewable energy over a relatively long time, power generating plants must be provided that can ensure that a basic demand is covered. The provision of power plant capacities that is necessary for this must be economically viable as a business proposition or possibly funded by state provisions, since in this case too there are on the one hand comparatively high fixed costs and on the other hand a relatively low operating time.

Conventional power generating plants, i.e. power plants that are based on fossil or biogenous energy carriers or nuclear energy, can provide electrical energy on a planned basis over a long time. However, for political reasons, in particular reasons of sustainability and environmental protection, the use of plants based on fossil energy carriers or nuclear power is increasingly to be reduced in favor of electricity generators that are based on renewable energy sources. However, these electricity generators must be installed in relation to demand and for their part be able to be operated economically. As from a certain degree of installed capacity on the basis of renewable energy sources, it is economically more advisable to install storage capacity instead of further increasing renewable energy capacities, so that, at times when there is an excess of electricity from renewable energy, it can be appropriately used and stored and, at times when there is a shortfall of electricity, electricity can be provided from energy stores or conventional power generating plants. If energy consumption is expediently made more flexible, it can be assumed here that the times when there is a noticeable surplus or shortfall of electricity will become less. For these short times there is in spite of everything the necessity to safeguard the electricity supply, while accomplishing this as economically as possible.

In view of the prior art, it is thus an object of the present invention to provide an improved plant that is not affected by the disadvantages of conventional methods.

In particular, it was an object of the present invention to find ways of making it possible to increase the flexibility with regard to the storage and use of electrical energy in comparison with the prior art.

Furthermore, the plant should allow for flexible operation, so that it is possible to respond particularly flexibly to any change in the electricity supply and/or demand, in order for example to achieve economic advantages. At the same time, it should be possible for the plant to be used for storing or providing electrical energy even over relatively long periods of a high or low electricity supply.

Furthermore, the security of supply should be improved by the present invention.

The plant and the method should also have the highest possible efficiency. Furthermore, the method according to the invention should allow itself to be carried out using infrastructure that is conventional and widely available.

In addition, the method should allow itself to be carried out with the fewest possible method steps, but they should be simple and reproducible.

Further objects that are not explicitly mentioned arise from the overall context of the following description and the claims.

These and further objects that are not expressly mentioned and arise from the circumstances discussed at the beginning are achieved by an integrated plant in which a plant for the electrothermic production of hydrogen cyanide, a separating device for separating hydrogen cyanide from the reaction mixture of the electrothermic production of hydrogen cyanide and a device for introducing a gas into a natural gas network are connected such that a stream of gas containing hydrogen and/or hydrocarbons can be introduced from the separating device into the natural gas network.

The subject matter of the present invention is accordingly an integrated plant which comprises a plant for the electrothermic production of hydrogen cyanide and a separating device for separating hydrogen cyanide from the reaction mixture of the electrothermic production of hydrogen cyanide while obtaining at least one stream of gas containing hydrogen and/or hydrocarbons, the integrated plant having a device for introducing a gas into a natural gas network, to which device a stream of gas containing hydrogen and/or hydrocarbons is fed from the separating device via at least one conduit.

The subject matter of the present invention is also a method for the flexible use of electricity in an integrated plant according to the invention in which a stream of gas, containing hydrogen and/or hydrocarbons, is fed into a natural gas network and the amount and/or the composition of the stream of gas fed into the natural gas network is changed in dependence on the electricity supply.

The integrated plant according to the invention and the method according to the invention have a particularly good range of properties, while the disadvantages of conventional methods and plants can be reduced significantly.

In particular, it has been found in a surprising way that it is thereby possible to operate a plant for the electrothermic production of hydrogen cyanide with a high degree of utilization, while renewable energy sources can be economically used when there is a surplus. Furthermore, the plant allows a surplus of electricity from renewable energy sources, including wind power or photovoltaics, to be converted into a storable form.

Furthermore, electrical energy can also be provided in a particularly low-cost way when there is a relatively long period of a low supply of renewable energy.

A plant for the electrothermic production of hydrogen cyanide can be operated well dynamically, and can therefore be adapted variably to the electricity supply. At the same time, the integrated plant can be used for storing or providing electrical energy even over relatively long periods of a high or low electricity supply. At the same time, surprisingly long runtimes of all the components of the integrated plant can be achieved, so that their operation can be made very economical.

It may also be provided that the plant for the electrothermic production of hydrogen cyanide is of a controllable design, the control being performed in dependence on the electricity supply.

In a preferred embodiment of the method according to the invention, electricity from renewable energy sources is used for the electrothermic production of hydrogen cyanide.

In addition, the method can be carried out with relatively few method steps, these being simple and reproducible.

The use of electricity from renewable energy sources enables the present integrated plant to provide chemical derivatives with little release of carbon dioxide, since the hydrogen cyanide obtained can be converted into many chemically important derivatives with very high conversion rates and, in comparison with alternative starting materials, with less further energy being supplied or greater release of heat.

The integrated plant according to the invention serves for the expedient and flexible use of electrical energy, also synonymously referred to herein as electricity. The integrated plant can store electrical energy when there is a high electricity supply and feed electrical energy into an electricity network in particular when there is a low electricity supply. The term storage refers here to the capability of the plant to transform electricity into a storable form, in the present case chemical energy, when there is a high supply of electricity, while this chemical energy can be converted into electrical energy when there is a low supply of electricity. The storage may in this case take place in the form of the co-product hydrogen, which inevitably occurs in the electrothermic production of hydrogen cyanide from methane or higher hydrocarbons. The storage may also take place in the form of products that can form in the electrothermic production of hydrogen cyanide, in an endothermic conversion taking place in parallel with the formation of hydrogen cyanide, for example by a conversion of two molecules of methane to ethane and hydrogen. It should be noted in this connection that two moles of methane (CH₄) have a lower energy content than for example one mole of ethane (C₂H₆) and one mole of hydrogen, so that energy can be stored by a conversion of methane into hydrogen and a hydrocarbon with two or more carbon atoms.

In conventional plants for the production of hydrogen cyanide, a relatively great amount of energy is expended on processing the secondary product gases occurring, in order to optionally sell hydrogen in pure form. In the present plant, this purification can be made very much easier by using the byproduct gases for their energy.

The integrated plant according to the invention comprises a plant for the electrothermic production of hydrogen cyanide. The term electrothermic refers in this case to a method in which hydrogen cyanide is produced in an endothermic reaction from hydrocarbons or coal and the heat required for carrying out the reaction is produced by electrical power. Preferably, gaseous or vaporized hydrocarbons are used, with particular preference aliphatic hydrocarbons. Particularly suitable are methane, ethane, propane and butane, in particular methane. In the electrothermic production of hydrogen cyanide from aliphatic hydrocarbons, hydrogen is obtained as a co-product.

The electrothermic preparation of hydrogen cyanide can be carried out by reacting hydrocarbons with ammonia or nitrogen in an arc reactor. The electrothermic preparation of hydrogen cyanide can be carried out in a single-stage process in which a gas mixture containing ammonia and at least one hydrocarbon is passed through the arc. As an alternative, a gas mixture comprising nitrogen and a hydrocarbon which may additionally contain hydrogen can also be passed through the arc. Suitable plants and processes for a single-stage electrothermic preparation of hydrogen cyanide in an arc are known from GB 780,080, U.S. Pat. No. 2,899,275 and U.S. Pat. No. 2,997,434. As an alternative, the electrothermic preparation of hydrogen cyanide can be carried out in a two-stage process in which nitrogen is passed through the arc and at least one hydrocarbon is fed downstream of the arc into the plasma produced in the arc. A suitable plant and a process for a two-stage electrothermic preparation of hydrogen cyanide are known from U.S. Pat. No. 4,144,444.

The arc reactor is preferably operated with an energy density of 0.5 to 10 kWh/Nm³, particularly 1 to 5 kWh/Nm³ and in particular 2 to 3.5 kWh/Nm³, the energy density relating to the volume of gas that is passed through the arc.

The temperature in the reaction zone of the arc reactor varies on the basis of the gas flow, it being possible for up to 20 000° C. to be reached in the center of the arc and the temperature to be about 600° C. at the periphery. At the end of the arc, the average temperature of the gas is preferably in the range from 1300 to 3000° C., with particular preference in the range from 1500 to 2600° C.

The desired production capacity is generally achieved by a parallel arrangement of a plurality of arc reactors which can be controlled jointly or separately.

The residence time of the feedstock in the reaction zone of the arc reactor is preferably in the range from 0.01 ms to 20 ms, with particular preference in the range from 0.1 ms to 10 ms and with special preference in the range from 1 to 5 ms. After that, the gas mixture emerging from the reaction zone is quenched, i.e. subjected to very rapid cooling to temperatures of less than 250° C., in order to avoid decomposition of the thermodynamically unstable intermediate product hydrogen cyanide. A direct quenching process, such as for example the feeding in of hydrocarbons and/or water, or an indirect quenching process, such as for example rapid cooling in a heat exchanger with steam generation, may be used for the quenching. Direct quenching and indirect quenching may also be combined with each other.

In a first embodiment, the gas mixture emerging from the reaction zone is only quenched with water. This embodiment features relatively low investment costs. However, it is disadvantageous that in this way a considerable part of the energy contained in the product gas is not used, or is used only with a low exergetic value.

In a second embodiment, the gas mixture emerging from the reaction zone is mixed with a hydrocarbon-containing gas or a hydrocarbon-containing liquid, at least some of the hydrocarbons being cracked endothermically. Depending on how the process is conducted, a more or less wide range of products is thereby produced, for example not only hydrogen cyanide and hydrogen but also fractions of ethane, propane, ethene and other lower hydrocarbons. This allows the heat produced to be passed on for further use, such as the endothermic cracking of the hydrocarbons, to a much greater extent.

After this lowering of the temperature, for example to 150 to 300° C., solid constituents, in particular carbon particles, are separated and the gas mixture, which may, depending on the starting materials, contain not only hydrogen cyanide and hydrogen but also further substances, such as ethyne, ethene, ethane, carbon monoxide and volatile sulphur compounds, such as H₂S and CS₂, is passed on for further processing to obtain hydrogen cyanide.

In an embodiment alternative to an arc reactor, the electrothermic preparation of hydrogen cyanide is carried out by reacting hydrocarbons with ammonia in an electrically heated fluidized bed of coke according to the Shawinigan process.

In a further alternative embodiment, the electrothermic preparation of hydrogen cyanide is carried out by reacting hydrocarbons with ammonia in the presence of a platinum-containing catalyst in the BMA process with electric heating of the reactor. The electric heating can be effected by resistance heating, for example as described in WO 2004/091773, by electric induction heating, for example as described in WO 95/21126, or by microwave heating, for example as described in U.S. Pat. No. 5,529,669 and U.S. Pat. No. 5,470,541.

The integrated plant according to the invention also comprises a separating device for separating hydrogen cyanide from the reaction mixture of the electrothermic production of hydrogen cyanide while obtaining at least one stream of gas containing hydrogen and/or hydrocarbons, and also a device for introducing a gas into a natural gas network, to which device at least one stream of gas containing hydrogen and/or hydrocarbons is fed from the separating device via a conduit.

In the separating device, hydrogen cyanide is separated from hydrogen and other hydrocarbons. Hydrogen cyanide may in this case be separated from the gas mixture by selective absorption into water. Ethyne formed along with hydrogen cyanide may be separated subsequently from the gas mixture by selective absorption into a solvent. Suitable solvents are, for example, water, methanol, N-methyl pyrrolidone or mixtures thereof. Suitable methods for the separation of hydrogen cyanide and ethyne from the gas mixture are known from the prior art, for example from Ullmann's Encyclopedia of Industrial Chemistry, 2012 Wiley-VCH Verlag GmbH & Co. KGaA, Weinheim, Volume 10, pages 675 to 678, DOI: 10.1002/14356007.a08_159.pub3 and Volume 1, pages 291 to 293, 299 and 300, DOI: 10.1002/14356007.a01_097.pub4.

The mixture separated from hydrogen cyanide and containing hydrogen and hydrocarbons can be fed directly to the device for introducing a gas into a natural gas network. Alternatively, hydrogen may be separated from the mixture separated from hydrogen cyanide and either hydrogen or a thereby resultant hydrocarbon-containing gas is fed to the device for introducing a gas into a natural gas network. Similarly, hydrogen and a hydrocarbon-containing gas may also be fed via separate conduits from the separating device for separating hydrogen cyanide from the reaction mixture of the electrothermic production of hydrogen cyanide to the device for introducing a gas into a natural gas network. The separation of hydrogen and hydrocarbons may in this case also take place incompletely, without incomplete separation having disadvantageous effects on the operation of the plant, so that the expenditure on apparatus and the energy consumption for the separation can be reduced in comparison with complete separation.

The device for introducing a gas into a natural gas network is not subject to any particular restrictions. All devices with which hydrogen, alkanes and alkenes can be introduced in gaseous form individually or mixed into a natural gas network are suitable.

In a preferred embodiment, the device for introducing a gas into a natural gas network comprises at least one reservoir for hydrogen. The type of reservoir is not critical, and so a pressurized tank, a liquefied gas reservoir, a reservoir with gas adsorption on a solid or a chemical reservoir, in which hydrogen is stored by a reversible chemical reaction, may be used for this. The capacity of the reservoir is preferably dimensioned to hold the amount of hydrogen produced by the plant for the electrothermic production of hydrogen cyanide at full load within 2 hours, with particular preference the amount produced within 12 hours and with most particular preference the amount produced within 48 hours. The use of a relatively large hydrogen reservoir permits hydrogen to be fed into the natural gas network over an extended period of time, allowing a maximum content of hydrogen in the natural gas network that is prescribed by the network operator to be maintained. Many end devices connected to the natural gas network can only be safely operated within a comparatively narrow band in respect of what is known as the Wobbe index, and a widening of the band would require complex additional installations on the end devices. The Wobbe index describes the combustion properties of natural gas. Feeding of hydrogen into the natural gas generally leads to a lowering of the Wobbe index. The Technical Rules, Worksheet G 260 of the DVGW stipulate lower limits for the Wobbe index. Depending on the composition of the natural gas, these limits may be reached even when just a few percent by volume of hydrogen is fed into the natural gas network.

The device for introducing a gas into a natural gas network preferably comprises at least one reservoir for a hydrocarbon-containing gas. A pressurized tank, a liquefied gas reservoir, a reservoir in which the hydrocarbons are absorbed in a solvent, or a reservoir with gas adsorption on a solid may be used as the reservoir. The capacity of the reservoir is preferably dimensioned to hold the amount of gaseous hydrocarbons produced by the plant for the electrothermic production of hydrogen cyanide at full load within 2 hours, with particular preference the amount produced within 12 hours and with most particular preference the amount produced within 48 hours.

The device for introducing a gas into a natural gas network preferably comprises a device for mixing gases. The device for mixing gases is preferably configured such that the Wobbe index, the calorific value or the density of the gas that is introduced into the natural gas network, or a combination of these gas properties, can be set. With particular preference, the device for mixing gases comprises a measuring device for determining the Wobbe index, calorific value or density of the mixed gas, with which the mixture of the gases can be controlled. With particular preference, the device for mixing gases is connected to a reservoir for hydrogen, and in a further preferred embodiment additionally also to a reservoir for a hydrocarbon-containing gas.

In a preferred embodiment, the device for introducing a gas into a natural gas network comprises a methanation reactor for reacting hydrogen with carbon dioxide or carbon monoxide to methane. In another preferred embodiment, the device for introducing a gas into a natural gas network comprises a Fischer-Tropsch reactor for reacting hydrogen and carbon monoxide to hydrocarbons. In a likewise preferred embodiment, the device for introducing a gas into a natural gas network comprises a hydrogenation reactor for reacting hydrogen and unsaturated hydrocarbons to saturated hydrocarbons. Suitable methanation reactors, Fischer-Tropsch reactors and hydrogenation reactors are known to a person skilled in the art from the prior art. All three embodiments provide a conversion of hydrogen to products of which the density, volume-specific calorific value and Wobbe index are higher than those of hydrogen. If hydrocarbons with at least 2 carbon atoms are generated, the density, the volume-specific calorific value and the Wobbe index are even above those of natural gas. Together with such hydrocarbons, hydrogen can be fed into the natural gas network in relatively great proportions without going below the limits for the density, calorific value and the Wobbe index prescribed by the codes of practice.

The integrated plant according to the invention preferably additionally comprises a plant for electricity generation, to which at least one stream of gas containing hydrogen and/or hydrocarbons is fed from the separating device via a conduit. Suitable here as plants for electricity generation are all plants with which electrical power can be generated from the stream of gas. Preferably, a plant for electricity generation that has a high efficiency is used.

In a preferred embodiment, the plant for electricity generation comprises a fuel cell. In this embodiment, the plant for electricity generation is preferably fed a stream of gas that substantially consists of hydrogen.

In a further preferred embodiment, the plant for electricity generation comprises a power generating plant with a turbine. With particular preference, the plant comprises a gas turbine that can be operated with hydrogen and/or hydrocarbon-containing gases. Used with most preference is a gas turbine that can be operated with mixtures of hydrogen and hydrocarbon-containing gases of changing composition.

Preferably, the power generating plant with a turbine is a gas-and-steam turbine power plant, also known as a combined cycle gas-and-steam power plant. In these power generating plants, the principles of a gas turbine power plant and a steam power plant are combined. A gas turbine generally serves here inter alia as a heat source for a downstream waste heat boiler, which in turn acts as a steam generator for the steam turbine.

In addition to the stream of gas fed from the separating device, the plant for electricity generation may also be fed further substances, for example additional hydrogen for the operation of a fuel cell or additional fuel for the operation of a turbine or the heating of a steam generator.

The power output of the plant for electricity generation may be chosen depending on the production capacity of the plant for the electrothermic production of hydrogen cyanide. Preferably, the output of the plant for electricity generation is chosen such that the power requirement of the plant for the electrothermic production of hydrogen cyanide at full load is completely covered by the plant for electricity generation. The power can in this case be achieved by a single device or a combined group of multiple devices, where the combined group (pool) can be achieved by way of a common control system. Electrical energy for the plant for the electrothermic production of hydrogen cyanide can also be drawn from the electricity network. Similarly, the plant for electricity generation may be dimensioned such that, in addition to the plant for the electrothermic production of hydrogen cyanide, further electricity consumers are also supplied or the electrical energy surplus to the requirements of the plant for the electrothermic production of hydrogen cyanide is fed into an electricity network.

In a particularly preferred embodiment of the integrated plant according to the invention, the plant for the electrothermic production of hydrogen cyanide comprises a steam generator, with which steam is generated from the waste heat of the electrothermic process, the plant for electricity generation comprises a device in which electricity is generated from steam, and the integrated plant comprises a steam conduit, with which steam generated in the steam generator is fed to the device in which electricity is generated from steam. Preferably, an indirect quenching of the reaction gas obtained in a reactor for producing hydrogen cyanide is used as the steam generator. The device in which electricity is generated from steam is preferably a steam turbine or a steam motor and with particular preference a steam turbine. With most preference, the steam turbine is part of a gas-and-steam turbine power plant. With this embodiment, waste heat generated in the plant for producing hydrogen cyanide can be used for generating electricity and the fuel requirement for operating the device in which electricity is generated from steam can be reduced.

In a preferred embodiment, the integrated plant according to the invention additionally comprises a reservoir for hydrogen cyanide. This reservoir makes it possible to continue operating downstream reactions for converting hydrogen cyanide into further products continuously, even when, at low electricity supply, only a little or no hydrogen cyanide at all is produced in the plant for the electrothermic production of hydrogen cyanide. The storage of hydrogen cyanide is preferably in liquid form.

In a further preferred embodiment, the integrated plant according to the invention is connected to a weather forecasting unit. Such a connection to a weather forecasting unit makes it possible to adapt the operation of the plant so as on the one hand to be able to make use of the possibility of using inexpensive surplus electricity and the possibility of providing electricity from the plant for electricity generation when there is a low electricity supply, and accordingly a high price for electricity, and on the other hand always to provide sufficient hydrogen cyanide for the continuous operation of a downstream, hydrogen cyanide-consuming plant. It is thus possible, depending on the result of the weather forecast, for example to bring a reservoir for hydrogen cyanide to a high or low filling level. In addition, a plant for the further processing of the hydrogen cyanide may be prepared and set up for modified operating modes. For instance, when there is a relatively long-term shortfall of electricity, these parts of the system can be set up for a reduced production capacity, so that an interruption in the operation owing to a lack of hydrogen cyanide can be avoided.

In addition, the integrated plant may be connected to a unit for producing a consumption forecast, where this unit has with preference a data memory which comprises data on historical consumption. The data on historical consumption may comprise for example the daily variation, the weekly variation, the annual variation and further variations in terms of the electricity demand and/or the electricity generation. The data on the consumption forecast may also take into consideration specific changes, for example the gain or loss of a major consumer. In addition or as an alternative, the data memory may also contain data on the historical variation in electricity prices.

The method according to the invention for the flexible use of electricity is carried out in an integrated plant according to the invention and a stream of gas, containing hydrogen and/or hydrocarbons, is fed into a natural gas network from the device for introducing a gas into a natural gas network. In this case, the integrated plant is operated such that the amount and/or the composition of the stream of gas fed into the natural gas network is changed in dependence on the electricity supply. This way, one can adjust the amount of electrical energy that is stored in the natural gas network in the form of chemical energy by generating or modifying the gas fed into the natural gas network.

The electricity supply may take the form both of a surplus of electricity and a shortfall of electricity. A surplus of electricity is obtained if at a certain time more electricity from renewable energy sources is provided than the total consumption of electricity at this time. A surplus of electricity is also obtained if large amounts of electrical energy from fluctuating renewable energy sources are provided and the cutting back or shutting down of power generating plants involves high costs. An electricity shortfall is obtained if comparatively small amounts from renewable energy sources are available and inefficient power generating plants or power generating plants involving high costs have to be operated. The cases of a surplus of electricity and shortfall of electricity described here may become evident in various ways. For example, the prices on the electricity exchanges may be an indicator of the respective situation, a surplus of electricity leading to lower electricity prices and a shortfall of electricity leading to higher electricity prices. A surplus of electricity or shortfall of electricity may, however, also exist without there being any direct effect on the electricity price. For example, a surplus of electricity may also exist if the operator of a wind farm produces more power than it has predicted and sold. By analogy, there may be a shortfall of electricity if the operator produces less power than it has predicted. According to the invention, the terms surplus of electricity and shortfall of electricity cover all of these cases.

In a first embodiment of the method according to the invention for the flexible use of electricity, in an integrated plant according to the invention, the plant for the electrothermic production of hydrogen cyanide is operated in dependence on the electricity supply, at least one stream of gas, containing hydrogen and/or hydrocarbons, is fed from the separating device to the device for introducing a gas into a natural gas network and a stream of gas, containing hydrogen and/or hydrocarbons, is fed into a natural gas network from the device for introducing a gas into a natural gas network.

In this first embodiment, the plant for the electrothermic production of hydrogen cyanide preferably comprises a number of reactors arranged in parallel and, in dependence on the electricity supply, all, only some or none of the reactors are operated. Preferably, the reactors are operated for this purpose under constant, optimized reaction conditions and the adaptation of the plant operation to the electricity supply is performed only by shutting down or starting up reactors. In an alternative preferred embodiment, individual reactors or all of the reactors are operated with variable throughputs and correspondingly variable electricity consumption.

In a second embodiment of the method according to the invention for the flexible use of electricity, in an integrated plant according to the invention which comprises a plant for electricity generation, to which a stream of gas containing hydrogen and/or hydrocarbons is fed from the separating device via a conduit, the ratio of the amount of gas that is fed from the separating device to the device for introducing a gas into a natural gas network and the amount of gas that is fed from the separating device to the plant for electricity generation is changed in dependence on the electricity supply and a stream of gas, containing hydrogen and/or hydrocarbons, is fed into a natural gas network from the device for introducing a gas into a natural gas network.

Preferably, in this embodiment the ratio of the amounts is changed such that, when there is a higher electricity supply, a greater proportion of the gas is fed into the natural gas network. With particular preference, when there is a moderate electricity supply, the gas from the separating device is fed completely or for the most part, in particular over 80%, to the plant for electricity generation and, when there is a high electricity supply, the plant for electricity generation is taken out of operation and the gas from the separating device is fed completely or for the most part, in particular over 80%, into the natural gas network.

The second embodiment of the method according to the invention makes it possible for the plant for the electrothermic production of hydrogen cyanide to be operated uniformly both when there is a moderate electricity supply and when there is a high electricity supply, resulting in a high level of plant utilization for this plant. The reduction of the electricity generation in the integrated plant and the introduction of gas into the natural gas network allows electrical energy to be additionally used when there is a high electricity supply, and effectively stored in the natural gas network in the form of chemical energy.

In a third embodiment of the method according to the invention for the flexible use of electricity, in an integrated plant according to the invention in which the plant for the electrothermic production of hydrogen cyanide comprises an arc reactor, the gas mixture emerging from the arc reactor is mixed with a hydrocarbon-containing gas or a hydrocarbon-containing liquid for cooling, the type and/or amount of the gas and/or the liquid being changed in dependence on the electricity supply, at least one stream of gas, containing hydrogen and/or hydrocarbons, is separated from the thereby resultant reaction mixture in the separating device and fed to the device for introducing a gas into a natural gas network and a stream of gas, containing hydrogen and/or hydrocarbons, is fed into a natural gas network from the device for introducing a gas into a natural gas network.

Preferably, in this embodiment, when there is a higher electricity supply, the gas mixture emerging from the arc reactor is mixed with a greater amount of hydrocarbon-containing gas or liquid or the type of the gas and/or the liquid is changed such that a greater part of the thermal energy of the gas mixture emerging from the arc reactor is used for the endothermic cracking of constituents of the gas and/or the liquid.

The hydrocarbons obtained by the endothermic cracking may be fed completely to the device for introducing a gas into a natural gas network. Alternatively, only a fraction may be fed to the device for introducing a gas into a natural gas network and the rest is fed as a feedstock for the production of hydrogen cyanide to the plant for the electrothermic production of hydrogen cyanide.

The features of the first and second embodiments described above of the method according to the invention may also be used in combination with each other. Preferably, then, when there is a low electricity supply, the plant for the electrothermic production of hydrogen cyanide is operated in dependence on the electricity supply and, when there is a high electricity supply, the ratio of the amount of gas that is fed from the separating device to the device for introducing a gas into a natural gas network and the amount of gas that is fed from the separating device to the plant for electricity generation is changed. With particular preference, the method according to the invention comprises the steps of
a) setting a first threshold value and a second threshold value for an electricity supply,
b) determining the electricity supply,
c) changing the proportion of gas that is fed from the separating device to the plant for electricity generation and the electrical power output of the plant for electricity generation in dependence on the electricity supply if the electricity supply exceeds the first threshold value and changing the output of the plant for the electrothermic production of hydrogen cyanide in dependence on the electricity supply if the electricity supply is below the second threshold value, and
d) repeating steps b) and c).

The threshold values are preferably set depending on the filling level of the reservoir for hydrogen cyanide at the particular time or depending on the predictions for the development of the consumption and generation of hydrogen cyanide in the next hours. If, for example, the filling level of the reservoir for hydrogen cyanide falls to a low value, the threshold value below which the output of the plant for the electrothermic production of hydrogen cyanide is reduced is set to a lower value.

The electricity supply may be determined either directly by agreement with electricity generators and/or electricity consumers or indirectly by way of trading platforms and/or by OTC methods and an associated electricity price. In a preferred embodiment, the electricity supply is determined by agreement with generators of electricity from wind energy and/or solar energy. In a further preferred embodiment, the electricity supply is determined by way of the electricity price on a trading platform.

If the electricity supply is determined by agreement with generators of electricity from wind energy and/or solar energy, preferably the electrical power output of the plant for electricity generation is changed in accordance with the surplus of electricity when the first threshold value is exceeded and the output of the plant for the electrothermic production of hydrogen cyanide is changed in accordance with the shortfall of electricity when the second threshold value is not reached.

If the electricity supply is determined by way of the electricity price on a trading platform, preferably the electrical power output of the plant for electricity generation is changed to a predetermined lower value when the first threshold value is exceeded and the output of the plant for the electrothermic production of hydrogen cyanide is changed to a predetermined lower value when the second threshold value is not reached.

Joint operation of the plant for the electrothermic production of hydrogen cyanide and the plant for electricity generation when there is a moderate electricity supply surprisingly allows high operating times to be attained, so that a high level of profitability of the plant is achieved.

Similarly, the features of the second and third embodiments may also be combined with each other. Used particularly advantageously for this purpose is an integrated plant in which the plant for the electrothermic production of hydrogen cyanide comprises a steam generator, with which steam is generated from the waste heat of the electrothermic process, and the plant for electricity generation comprises a steam turbine, which is driven with this steam. By changing the type and/or amount of the gas and/or the liquid that is used for cooling the gas mixture emerging from the arc reactor, it is then possible to adjust which fraction of the heat stemming from the electrothermic production of hydrogen cyanide is used directly in the steam turbine for electricity generation and which fraction is stored in the natural gas network in the form of chemical energy with the products of the endothermic cracking.

Finally, the features of the first and third embodiments or the features of all three embodiments may also be used in combination with one another.

In a preferred embodiment of the method according to the invention, the device for introducing a gas into a natural gas network comprises a reservoir for hydrogen and, from this reservoir, hydrogen is introduced into a natural gas pipeline, the amount of hydrogen introduced being set in dependence on the gas flow in the natural gas pipeline such that the Wobbe index, the calorific value or the density of the gas in the natural gas pipeline or a combination of these gas properties is kept within predetermined limits. The introduction of the hydrogen may be controlled by measuring these gas properties in the natural gas pipeline after introducing the gas that is to be introduced into the natural gas network.

Similarly, the device for introducing a gas into a natural gas network may also comprise a reservoir for a gas mixture of hydrogen and hydrocarbon-containing gases and the gas mixture may be introduced into a natural gas pipeline from this reservoir, the amount of the gas mixture introduced being set in dependence on the gas flow in the natural gas pipeline such that the Wobbe index, the calorific value or the density of the gas in the natural gas pipeline or a combination of these gas properties is kept within predetermined limits.

In a further preferred embodiment of the method according to the invention, the device for introducing a gas into a natural gas network comprises separate reservoirs for hydrogen and hydrocarbon-containing gases and a device for mixing gases that is connected to these reservoirs. In the device for mixing gases, hydrogen and hydrocarbon-containing gases are mixed, the ratio of the amounts being set such that the Wobbe index, the calorific value or the density of the resultant gas mixture or a combination of these gas properties is kept within predetermined limits. Preferably, hydrocarbons with two or more carbon atoms, in particular ethane, ethene, propane, propene, butane and/or butene, that have been separated in the separating device for separating hydrogen cyanide from the reaction mixture of the electrothermic production of hydrogen cyanide are used as hydrocarbon-containing gases. The gas mixture resulting after setting the gas properties is then fed into the natural gas network. Preferably, the Wobbe index of the resultant gas mixture is set such that the ratio of the Wobbe index of the gas fed into the natural gas network to the Wobbe index of the gas in the natural gas network lies in the range from 0.9:1 to 1:0.9, in particular in the range from 0.95:1 to 1:0.95.

Further gases, such as oxygen, nitrogen and preferably air, may additionally be admixed within the limits set by the applicable codes of practice for lowering the Wobbe index or setting other characteristic variables to the value prescribed by the operator of the gas pipeline.

In another preferred embodiment of the method according to the invention, the device for introducing a gas into a natural gas network comprises a methanation reactor for reacting hydrogen with carbon dioxide or carbon monoxide to methane, where a gas stream containing hydrogen is fed from the separating device to the methanation reactor and methane generated in the methanation reactor is fed into the natural gas network. The conversion of hydrogen into methane allows the restrictions for the feeding of hydrogen into a natural gas network to be avoided and also allows the gas to be fed into a natural gas network at a point with a low gas flow in the natural gas pipeline.

In an additional preferred embodiment of the method according to the invention, the device for introducing a gas into a natural gas network comprises a Fischer-Tropsch reactor for reacting hydrogen and carbon monoxide to hydrocarbons, where a stream of gas containing hydrogen is fed from the separating device to the Fischer-Tropsch reactor and gaseous hydrocarbons generated in the Fischer-Tropsch reactor are fed into the natural gas network.

In another preferred embodiment of the method according to the invention, the device for introducing a gas into a natural gas network comprises a hydrogenation reactor, where a stream of gas containing unsaturated hydrocarbons is fed from the separating device to the hydrogenation reactor and saturated hydrocarbons generated in the hydrogenation reactor are fed into the natural gas network. This embodiment can be used particularly advantageously when the electrothermic production of hydrogen cyanide is carried out in an arc reactor, the gas mixture emerging from the arc reactor is mixed with a hydrocarbon-containing gas or a hydrocarbon-containing liquid for cooling, unsaturated hydrocarbons being formed by cracking, a stream of gas containing hydrogen and unsaturated hydrocarbons is separated from the resultant gas mixture and this stream of gas is fed to the hydrogenation reactor. By hydrogenating the unsaturated hydrocarbons in this stream of gas, the hydrogen content of the stream of gas can be reduced without significant energy loss and a gas mixture that is better suited for feeding into a natural gas network can be obtained.

Preferably, in the case of the method according to the invention, the plant for the electrothermic production of hydrogen cyanide draws electricity from a gas power plant that is operated with gas from the natural gas network in dependence on the electricity supply. In this embodiment, the method according to the invention preferably comprises the steps of
a) setting a first threshold value and a second threshold value for an electricity supply,
b) determining the electricity supply,
c) changing the electrical power output of the gas power plant in dependence on the electricity supply if the electricity supply exceeds the first threshold value and changing the output of the plant for the electrothermic production of hydrogen cyanide in dependence on the electricity supply if the electricity supply is below the second threshold value, and
d) repeating steps b) and c).

The threshold values are preferably set depending on the filling level of the reservoir for hydrogen cyanide at the particular time or depending on the predictions for the development of the consumption and generation of hydrogen cyanide in the next hours. If, for example, the filling level of the reservoir for hydrogen cyanide falls to a low value, the threshold value below which the output of the plant for the electrothermic production of hydrogen cyanide is reduced is set to a lower value.

The electricity supply may be determined either directly by agreement with electricity generators and/or electricity consumers or indirectly by way of trading platforms and/or by OTC methods and an associated electricity price. In a preferred embodiment, the electricity supply is determined by agreement with generators of electricity from wind energy and/or solar energy. In a further preferred embodiment, the electricity supply is determined by way of the electricity price on a trading platform.

The absolute level of the first threshold value from which a reduction of the output of the plant for electricity generation takes place is not important for this embodiment of the present method and can be set on the basis of economic criteria. The same applies to the second predetermined value, below which a reduction of the output of the plant for the electrothermic production of hydrogen cyanide takes place. The first threshold value and the second threshold value are preferably chosen to be the same.

Preferably, when there is a high electricity supply, the electrical energy used for the production of hydrogen cyanide originates at least partially from renewable energy sources, with particular preference from wind power and/or solar energy. However, it should be noted that, according to current German legislation, electricity that has been obtained from renewable energy sources may be fed into the electricity network even without any demand at the particular time and must be paid for. Therefore, conventionally generated electricity may at times constitute a “surplus”, since it may be less profitable for a power plant operator to run a power plant down to a low output than to sell electricity below the cost price. This surplus electrical energy obtained from the continued operation of conventional plants can be economically used by the present method, in particular stored.

The electricity supply is preferably calculated in advance from the data of a weather forecast. On the basis of the electricity supply calculated in advance, the aforementioned threshold values for an electricity supply are then preferably chosen such that, in the time period of the forecast, on the one hand a planned amount of hydrogen cyanide is produced.

Further preferred embodiments of the method according to the invention arise from the description given above of an integrated plant according to the present invention.

The present integrated plant and the method are suitable for the production of hydrogen cyanide in a very economical and resource-conserving way. Hydrogen cyanide can be transformed into many valuable intermediate products, while it is possible in this way to achieve a surprising reduction in the carbon dioxide emissions.

This surprising reduction is based on a number of synergistically acting factors. These include the fact that electricity from renewable energy sources can be used for the production of hydrogen cyanide, allowing the production of hydrogen cyanide to be adapted very flexibly to an electricity supply. Furthermore, hydrogen can be obtained with a very high electricity efficiency, and can be used for generating electrical energy without the release of carbon dioxide. Furthermore, heat is often released in the production of the valuable derivatives. This waste heat can often be used to cover the heat requirement in other parts of the process (for example in the case of distillative separation processes). The emission of carbon dioxide is reduced correspondingly if, on the other hand, an oxidation of hydrocarbons were necessary to generate the process heat.

Furthermore, the hydrogen cyanide produced can be used for preparing sodium cyanide, acetone cyanohydrin or methionine.

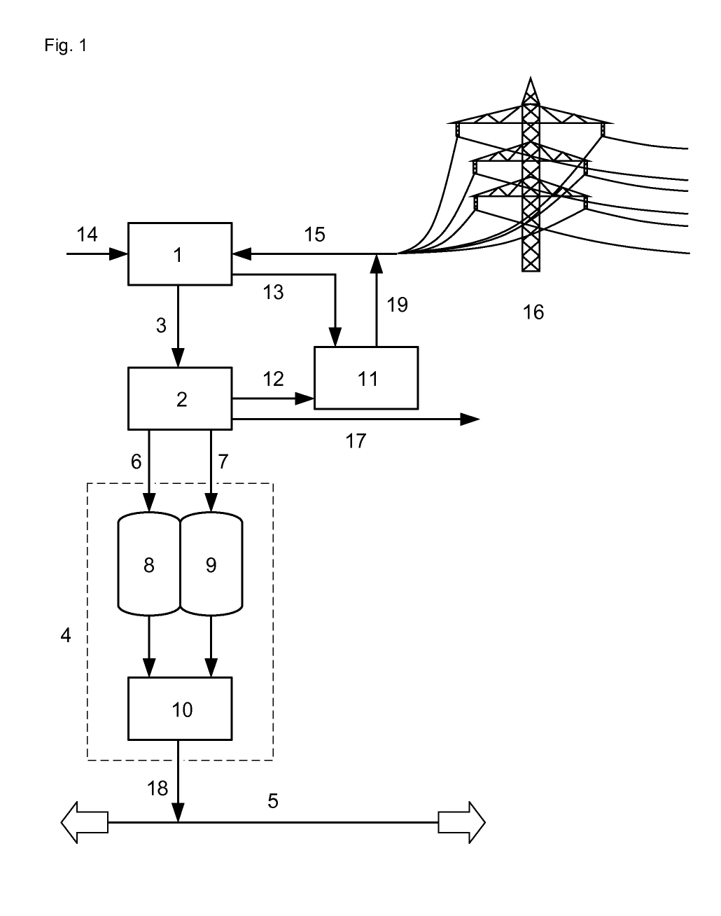

Preferred embodiments of the present invention are explained by way of example below on the basis of FIG. 1.

FIG. 1 schematically shows the structure of an integrated plant according to the invention, which comprises a plant 1 for the electrothermic production of hydrogen cyanide, a separating device 2 for separating hydrogen cyanide from the reaction mixture 3 of the electrothermic production of hydrogen cyanide and a device 4 for introducing a gas into a natural gas network 5.

In the plant 1 for the electrothermic production of hydrogen cyanide, hydrogen cyanide is generated from a hydrocarbon-containing starting material that is fed in via a conduit or a conveying element 14. Natural gas and lower hydrocarbons, in particular C₂-C₄ hydrocarbons, are suitable as the hydrocarbon-containing starting material. Nitrogen and ammonia are suitable as nitrogen-containing starting material.

Electrical power is drawn from an electricity network 16 via an electricity line 15 for the electrothermic production of hydrogen cyanide.

The reaction mixture 3 obtained in the electrothermic production of hydrogen cyanide is fed to a separating device 2, in which hydrogen cyanide is separated from the reaction mixture and is obtained as a product via a conduit 17. In the separation of hydrogen cyanide, hydrogen and optionally hydrocarbons, as well as further components, such as carbon black and sulphur-containing compounds, are separated. Hydrogen and hydrocarbons may be obtained in the separating device in the form of a stream of gas containing hydrogen and hydrocarbons. Preferably, however, a partial or complete separation of hydrogen and hydrocarbons is carried out and a hydrogen-rich stream of gas is fed via a conduit 6 and, separately, a hydrocarbon-rich stream of gas is fed via a conduit 7 to the device 4 for introducing a gas into a natural gas network.

From the device 4 for introducing a gas into a natural gas network, a gas containing hydrogen and/or hydrocarbons is introduced into a natural gas network 5 via a connecting conduit 18. In the preferred embodiment shown in FIG. 1, the device 4 for introducing a gas into a natural gas network additionally comprises a reservoir 8 for hydrogen, a reservoir 9 for a hydrocarbon-containing gas and a device 10 for mixing gas, with which hydrogen, hydrocarbon-containing gas and possibly further gases can be mixed, so that a stream of gas with a specifically set composition can be introduced into the natural gas network 5 via the connecting conduit 18.

In the embodiment shown in FIG. 1, the integrated plant also comprises a plant 11 for electricity generation, to which a stream of gas containing hydrogen and/or hydrocarbons is fed from the separating device 2 via a conduit 12. In the plant 11 for electricity generation, electricity is generated from the gases. This may take place by way of a combustion process, preferably in a gas-and-steam power plant, in which electricity is generated by gas and steam turbines. Alternatively, however, fuel cells may also be used for generating electricity from hydrogen and/or hydrocarbon-containing gas. The electricity generated in the plant 11 for electricity generation can be fed via the electricity lines 19 and 15 to the plant 1 for the electrothermic production of hydrogen cyanide and used for the electrothermic production of hydrogen cyanide. However, the electricity generated in the plant 11 for electricity generation may alternatively also be fed into the electricity network 16, in particular if the plant 1 for the electrothermic production of hydrogen cyanide is out of operation or consumes less electricity than is generated in the plant 11 for electricity generation.

As an alternative to the feeding of hydrogen and/or hydrocarbons from the separating device 2 via the conduit 12, hydrogen and/or hydrocarbons may also be fed to the plant 11 for electricity generation from the reservoirs 8 and/or 9 of the device 4 for introducing a gas into a natural gas network. The plant 11 for electricity generation may also be fed further fuel via a device that is not shown in FIG. 1.

In the embodiment shown in FIG. 1, the integrated plant additionally comprises a steam generator (not shown) in the plant 1 for the electrothermic production of hydrogen cyanide, a steam turbine (not shown) in the plant 11 for electricity generation, and a steam conduit 13, with which steam generated in the steam generator is fed to the steam turbine.

### LIST OF REFERENCE SIGNS

1 Plant for the electrothermic production of hydrogen cyanide
2 separating device for separating hydrogen cyanide
3 reaction mixture of the electrothermic production of hydrogen cyanide
4 device for introducing a gas into a natural gas network
5 natural gas network
6 conduit for a stream of gas containing hydrogen
7 conduit for a stream of gas containing hydrocarbons
8 reservoir for hydrogen
9 reservoir for a hydrocarbon-containing gas
10 device for mixing gases
11 plant for electricity generation
12 conduit for a gas stream containing hydrogen and/or hydrocarbons
13 steam conduit
14 conduit or conveying element for hydrocarbon-containing starting material
15 electricity line
16 electricity network
17 conduit for hydrogen cyanide
18 connecting conduit into the natural gas network
19 electricity line

1-27. (canceled)

28. An integrated plant, comprising an electrothermic hydrogen cyanide production plant and a separating device, separating hydrogen cyanide from the reaction mixture of the electrothermic production of hydrogen cyanide and providing at least one stream of gas containing hydrogen, hydrocarbons, or both, wherein the integrated plant comprises a device for introducing a gas into a natural gas network, to which device a stream of gas containing hydrogen, hydrocarbons, or both is fed from the separating device via at least one conduit.

29. The integrated plant of claim 28, wherein the device for introducing a gas into a natural gas network comprises at least one reservoir for hydrogen.

30. The integrated plant of claim 28, wherein the device for introducing a gas into a natural gas network comprises at least one reservoir for a hydrocarbon-containing gas.

31. The integrated plant of claim 28, wherein the device for introducing a gas into a natural gas network comprises a device for mixing gases.

32. The integrated plant of claim 28, wherein the device for introducing a gas into a natural gas network comprises a methanation reactor for reacting hydrogen with carbon dioxide or carbon monoxide to methane.

33. The integrated plant of claim 28, wherein the device for introducing a gas into a natural gas network comprises a Fischer-Tropsch reactor for reacting hydrogen and carbon monoxide to hydrocarbons.

34. The integrated plant of claim 28, wherein the device for introducing a gas into a natural gas network comprises a hydrogenation reactor for reacting hydrogen and unsaturated hydrocarbons to saturated hydrocarbons.

35. The integrated plant of claim 28, additionally comprising an electrical power plant, to which at least one stream of gas containing hydrogen, hydrocarbons, or both is fed from the separating device via a conduit.

36. The integrated plant of claim 35, wherein the electrothermic hydrogen cyanide production plant comprises a steam generator, with which steam is generated from the waste heat of the electrothermic process, the electrical power plant comprises a device in which electricity is generated from steam, and the integrated plant comprises a steam conduit, with which steam generated in the steam generator is fed to the device in which electricity is generated from steam.

37. The integrated plant of claim 35, wherein the electrical power plant is a gas-and-steam turbine power plant.

38. The integrated plant of claim 28, additionally comprising a connection to a weather forecasting unit.

39. The integrated plant of claim 28, wherein the electrothermic hydrogen cyanide production plant comprises an arc reactor.

40. The integrated plant of claim 28, wherein the electrothermic hydrogen cyanide production plant comprises a reactor having an electrically heated fluidized bed of coke.

41. The integrated plant of claim 28, wherein the electrothermic hydrogen cyanide production plant comprises an electrically heated reactor containing a platinum-containing catalyst.

42. A method for the flexible use of electricity, wherein an electrothermic production of hydrogen cyanide is carried out in an integrated plant according to claim 28; a stream of gas containing hydrogen, hydrocarbons, or both, is fed into a natural gas network from the device for introducing a gas into a natural gas network; and an amount, a composition, or both of said stream of gas fed into the natural gas network is changed depending on electricity supply.

43. The method of claim 42, wherein, in an integrated plant according to claim 28, the electrothermic hydrogen cyanide production plant is operated using an electricity supply; at least one stream of gas, containing hydrogen, hydrocarbons, or both, is fed from the separating device to the device for introducing a gas into a natural gas network; and a stream of gas containing hydrogen, hydrocarbons, or both, is fed into a natural gas network from the device for introducing a gas into a natural gas network.

44. The method of claim 42, wherein:
a) said integrated plant further comprises an electrical power plant, to which at least one stream of gas containing hydrogen, hydrocarbons, or both is fed from the separating device via a conduit; and
b) in said integrated power plant:
i) an electrothermic production of hydrogen cyanide is carried out;
ii) the ratio of the amount of gas that is fed from the separating device to the device for introducing a gas into a natural gas network and the amount of gas that is fed from the separating device to the plant for electricity generation is changed depending on electricity supply; and
ii) a stream of gas containing hydrogen, hydrocarbons, or both, is fed into a natural gas network from the device for introducing a gas into a natural gas network.

45. The method of claim 42, wherein:
a) the electrothermic hydrogen cyanide production plant in said integrated power plant comprises an arc reactor; and
b) in said integrated power plant:
i) an electrothermic production of hydrogen cyanide is carried out in the arc reactor;
ii) the gas mixture emerging from the arc reactor is mixed with a hydrocarbon-containing gas or a hydrocarbon-containing liquid for cooling, providing a reaction mixture, and the type, the amount, or both of said hydrocarbon-containing gas or hydrocarbon-containing liquid are changed depending on electricity supply;
iii) at least one stream of gas containing hydrogen, hydrocarbons, or both, is separated from said reaction mixture in the separating device and fed to the device for introducing a gas into a natural gas network; and
iv) a stream of gas, containing hydrogen, hydrocarbons, or both, is fed into a natural gas network from the device for introducing a gas into a natural gas network.

46. The method of claim 42, wherein the device for introducing a gas into a natural gas network comprises a reservoir for hydrogen and, from this reservoir, hydrogen is introduced into a natural gas pipeline, the amount of hydrogen introduced being set depending on the gas flow in the natural gas pipeline such that the Wobbe index, the calorific value or the density of the gas in the natural gas pipeline or a combination of these gas properties is kept within predetermined limits.

47. The method of claim 42, wherein the device for introducing a gas into a natural gas network comprises separate reservoirs for hydrogen and hydrocarbon-containing gases and a device for mixing gases that is connected to these reservoirs and, in the device for mixing gases, hydrogen and hydrocarbon-containing gases are mixed, the ratio of the amounts being set such that the Wobbe index, the calorific value or the density of the resultant gas mixture or a combination of these gas properties is kept within predetermined limits.

48. The method of claim 42, wherein the device for introducing a gas into a natural gas network comprises a methanation reactor for reacting hydrogen and carbon dioxide to methane, a stream of gas containing hydrogen is fed from the separating device to the methanation reactor and methane generated in the methanation reactor is fed into the natural gas network.

49. The method of claim 42, wherein the device for introducing a gas into a natural gas network comprises a Fischer-Tropsch reactor for reacting hydrogen and carbon monoxide to hydrocarbons, a stream of gas containing hydrogen is fed from the separating device to the Fischer-Tropsch reactor and gaseous hydrocarbons generated in the Fischer-Tropsch reactor are fed into the natural gas network.

50. The method of claim 42, wherein the device for introducing a gas into a natural gas network comprises a hydrogenation reactor, a stream of gas containing unsaturated hydrocarbons is fed from the separating device to the hydrogenation reactor and saturated hydrocarbons generated in the hydrogenation reactor are fed into the natural gas network.

51. The method of claim 42, wherein the electricity supply is calculated in advance from data of a weather forecast.

52. The method of claim 42, wherein the electrothermic hydrogen cyanide production plant draws electricity from a gas power plant that is operated with gas from the natural gas network depending on electricity supply.

53. The method of claim 52, comprising the steps of
a) setting a first threshold value and a second threshold value for an electricity supply;
b) determining the electricity supply;
c) changing the electrical power output of the gas power plant depending on the electricity supply if the electricity supply exceeds the first threshold value and changing the output of the plant for the electrothermic production of hydrogen cyanide in dependence on the electricity supply if the electricity supply is below the second threshold value; and
d) repeating steps b) and c).

54. The method of claim 53, wherein the first threshold value and the second threshold value are the same.
